(19) United States
(12) Patent Application Publication
(10) Pub. No.: US 2006/0241318 A1
(43) Pub. Date: Oct. 26, 2006

(54) Preparation of [18F]fluorine labeled aromatic L-amino acids

(76) Inventors: Hans-Juergen Machulla, Tuebingen (DE); Michael Uebele, Tuebingen (DE); Blocher Achim, Grenzach-Whylen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/388,793

(22) Filed: Mar. 24, 2006

Related U.S. Application Data
(63) Continuation of application No. PCT/EP04/10094, filed on Sep. 10, 2004.

(30) Foreign Application Priority Data
Sep. 25, 2003 (DE) 103 46 228.7

(51) Int. Cl. C07C 227/16

(52) U.S. Cl. 560/38; 534/11; 560/40

(57) ABSTRACT

A method for preparing [¹⁸F]fluorine labeled aromatic L-amino acids is described, whereby the labeling reaction occurs at an L-enantiomeric aromatic amino acid provided with a protecting group. Furthermore, a method for preparing a diagnostic agent is described, whereby the [¹⁸F]fluorine labeled aromatic L-amino acid is used as prepared according to the invention. In addition, a method for visualizing of metabolic processes is described, whereby the [¹⁸F]fluorine labeled aromatic L-amino acid as prepared according to the invention is introduced into a living organism. Furthermore, an L-enantiomeric labeling precursor is described.

### CROSS-REFERENCE TO RELATED APPLICATIONS

This application is a continuation of copending International Patent Application PCT/EP2004/010094 filed on Sep. 10, 2004 and designating the United States, which was published under PCT Article 21 (2) not in English, and claims priority of German Patent Application DE 103 46 228.7 filed on Sep. 25, 2003, which is incorporated herein by reference.

### BACKGROUND OF THE INVENTION

1. Field of the Invention

The present invention relates to a method for preparing [¹⁸F]fluorine labeled aromatic L-amino acids, a method for preparing a [¹⁸F]fluorine labeled diagnostic agent, and a method for visualizing metabolic processes.

2. Description of the Related Art

Radioactive labeled amino acids are used in various fields of the nature science, especially in the bio sciences, but also in medicine. This concerns especially to so-called “tracers”, i.e. amino acids by means of which biological, biochemical, chemical or similar processes can be traced. The labeling of the amino acids is performed by the incorporation of short-lived radionuclides (radio indicators or radio active tracers). An example for such a radionuclide is [¹⁸F]fluorine. The radioactive labeled amino acids or tracers, respectively, can be constructed such that they act in the same way like unlabeled substances. Furthermore, also a selected modification of the characteristics of the tracer in comparison to those of the corresponding unlabeled substances is possible that e.g. no or a precisely strong restricted metabolisation of the tracer occurs (“metabolic trapping”), when it is introduced into a living organism.

Such radioactive labeled amino acids are e.g. used as tracers in the positron emission tomography (PET). PET is a scintigraphic diagnostic method that combines the advantages of tomographic slice imaging with the selective visualization of physiological metabolic functions. By means of PET it is possible to visualize essential metabolic processes in vivo. The tracers which are used in the PET release positrons during their disintegration at the place of their in vivo biological activity, which finally results in the emission of gamma rays. This radioactive radiation can be detected qualitatively as well as quantitatively by the aid of a PET scanner and, thus, enables the drawing of conclusions in view of alterations in the metabolic system of a patient.

The most important diagnostic fields of PET are the analysis of local blood flow and oxygen consumption, substrate metabolism, enzyme concentrations, and the biochemistry of neural and hormonal transmitters, especially receptor densities and occupancies as well as immunoreactions. Furthermore, by means of PET also molecular processes of the protein biosynthesis can be analyzed.

PET is more and more used in diagnostics and in the supportive therapy control of heart and cancer diseases as well as neurodegenerative diseases, such as Parkinson's disease, Alzheimer's, dementia.

A further interesting approach exists in the field of pharmacology where pharmacologically effective amino acids are labeled radioactively and quantified in view of their distribution at the site of action and assayed in respect of their effectivity by means of PET.

Especially on account of the number of global PET centers which is strongly growing year by year (270 centers in the year 2001), as well as on account of their increasing medical-scientific acceptance, there is an increased need for tracers, e.g. for [¹⁸F]fluorine labeled aromatic L-amino acids.

In the art several methods for preparing of [¹⁸F]fluorine labeled aromatic L-amino acids 1 are known. The most common method in this connection is the electrophilic substitution via [¹⁸F]fluorine gas ([¹⁸F]F₂). With this synthesis a fluorine destannylation of the precursor 2 is performed as an electrophilic substitution in which [¹⁸F]F₂ is used:

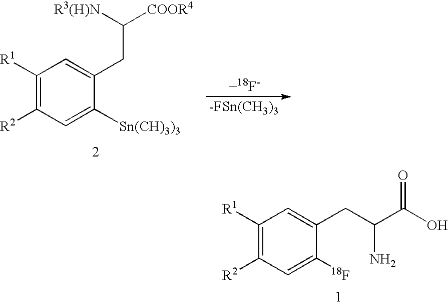

This known method is e.g. described in A. Luxen, M. Guillaume, W. P. Melega, V. W. Pike, O. Solin, R. Wagner, Nucl. Med. Biol, Vol. 19, No. 2, 149-158 (1992).

This method has several considerable disadvantages. [¹⁸F]F₂ is prepared via the radiation of neon, whereby 0.1% [¹⁹F]F₂ is mixed to the neon. For this reason supported labeled aromatic L-amino acids are obtained by the electrophilic substitution, i.e. such amino acids which are labeled with non-radioactive fluorine. Furthermore, only 50% of the fluorine-18-atoms can be used, half of it reacts with the leaving group. In addition, long time periods for the radiation of neon are necessary, however, the rate of yield of [¹⁸F]F₂ is poor.

Another important method is the nucleophilic substitution via [¹⁸F]fluorine, which starts at different precursors which are converted to the [¹⁸F]fluorine labeled aromatic L-amino acids by manually performed reactions. 6-[¹⁸F]fluoro-3,4-dihydroxy-L-phenylalanine (FDOPA) is e.g. produced from the nitrobenzaldehyde derivatives 3 (R¹═R²═OH-protecting group). These are converted after the labeling step with [¹⁸F]fluorine via a multi-stage synthesis to FDOPA:

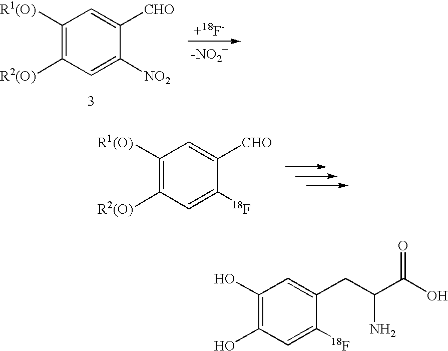

This known method is e.g. described in Lemaire C., Guillaume M., Cantineau R., Christiaens L., J. Nucl. Med. 31, 1247 (1990); Antoni G., Langstrom B., Acta Chem. Scand. 40, 152 (1987); Lemaire C., Guillaume M., Christiaens L., J. Nucl. Med. 30, 752 (1989); Lemaire C., Guillaume M., Cantineau R., Plenevaux A., Christiaens L., J. Label. Compds. Radiopharm. 30, 269 (1991).

This method has the decisive disadvantage that it comprises a multi-stage synthesis which is very labor-intensive, time consuming and costly. Furthermore, this known method provides a deficiently enantiomeric purity. In addition to a separation of products by means of a HPLC purification also a separation of the enantiomers by a chiral HPLC unit is required. The known method is therefore not well suitable for an automated routine production of [¹⁸F]fluorine labeled L-amino acids.

Other methods for preparing [¹⁸F]fluorine labeled aromatic L-amino acids are described in the art. E.g. 2-[¹⁸F]fluoro-L-phenylalanine (1, R¹═R²═H) or 2-[¹⁸F]fluoro-L-tyrosine (1, R¹═H, R²═OH) are produced by a technically very problematic fluorine dediazotation reaction from the corresponding triazene derivatives 4 (R⁴=alkyl groups) or via a nucleophile substitution at the trimethyl ammonium derivatives 5:

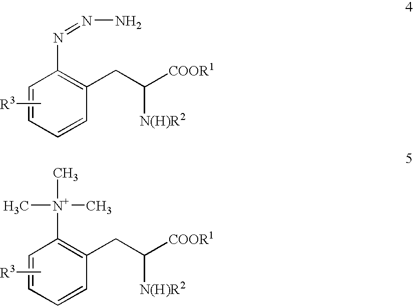

This known method is also described in the review article of Luxen et al. (l.c.). Details on the triazene derivative 4 is also described in Thierry Pages, Bernhard R. Langlois, Didier Le Bars, Patricia Landais, J. fluorine Chem. 107, 329-335 (2001). The trialkyl ammonium derivative 5 is described in WO 02/44144 A2.

Also these methods have several disadvantages. The methods described therein are associated with big technical problems so that an automized operation is not possible. A variety of side reactions requires a complex purification of the products. Furthermore, the highly interesting FDOPA cannot be produced via the two shown derivatives 4 and 5.

### SUMMARY OF THE INVENTION

Therefore, the problem underlying the invention is to provide a method for preparing a [¹⁸F]fluorine labeled aromatic L-amino acids, by means of which the aforesaid disadvantages can be avoided. Especially a method should be provided which is characterized by few synthesis steps, a short synthesis time period and a high reproducibility. Furthermore, such a method should be easily automatable.

This problem is solved by providing a method for preparing [¹⁸F]fluorine labeled aromatic L-amino acids comprising the following steps:

(1) providing the following L-enantiomeric compound in an appropriate reaction medium: 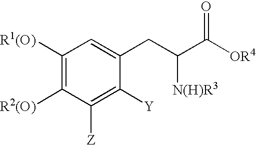
whereby R¹ and R² are appropriate protecting groups for OH groups, provided a hydroxy amino acid is to be prepared; whereby Z is an electron-attracting group; whereby Y is a leaving group for a nucleophilic substitution; whereby R³ is/are one or several appropriate protecting group(s) for an amino function; whereby R⁴ is an appropriate protecting group for a carboxyl group;

(2) nucleophilic substitution of Y for a negatively charged [¹⁸F]fluorine ion to prepare the following compound:

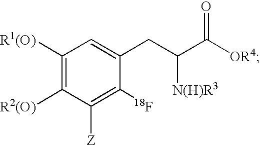

(3) cleaving-off of Z to prepare the following compound:

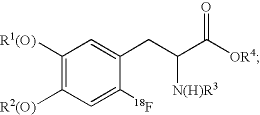

(4) hydrolytic cleaving-off of R³ and R⁴ to prepare the following compound:

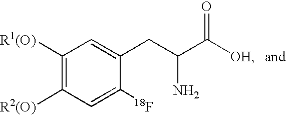

(5) hydrolytic cleaving-off of R¹ and R² to prepare the following [¹⁸F]fluorine labeled aromatic L-amino acid: 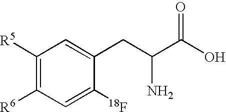
whereby R⁵ and R⁶ are substituents which are in each case an H atom or an OH group.

The problem underlying the invention is hereby completely solved.

The inventors have realized that when starting with the L-enantiomeric pure labeling precursor as provided in step (1) surprisingly in only few steps [¹⁸F]fluoro-L-phenylalanine derivatives, i.e. [¹⁸F]fluorine labeled aromatic amino acids such as 2-[¹⁸F]fluoro-L-phenylalanine (R⁵═R⁶═H), 2-[¹⁸F]fluoro-L-tyrosine (R⁵═H, R⁶═OH), 2-[¹⁸F]fluoro-5-hydroxy-L-phenylalanine (R⁵═OH, R⁶═H; 2-[¹⁸F]fluoro-meta-L-tyrosine) and 6-[¹⁸F]fluoro-3,4-dihydroxy-L-phenylalanine (R⁵═R⁶═OH; [¹⁸F]DOPA), can be prepared. It was especially surprising that it was possible at all to start from already chirally pure L-enantiomeric aromatic amino acids which were provided with protecting groups, to perform a direct labeling and conversion to the intended L-amino acid via a carrier-free [¹⁸F]fluorine within few steps. It is argued so far in the art that a labeling has always to be performed on simple amino acid precursors and only after this has been done the actual “construction” of the amino acid and a separation of the enantiomers can be done.

In the practical realization of the method according to the invention steps (4) and (5) take place simultaneously, i.e. the hydrolytic cleaving-off of the protecting groups R¹ to R⁴ takes place in a single step. Simply for a better illustration of the preparation of different labeled amino acids, i.e. of hydroxy and non-hydroxy amino acids, the distinction between two steps has been made.

It goes without saying that the protecting groups R¹ and R² only have to be provided in case that hydroxy amino acids, i.e. labeled tyrosine (only R²), hydroxy-phenylalanine (only R¹) or DOPA (R¹ and R²), should be prepared. For the preparation of non-hydroxy amino acids R^{1/2}(O) is replaced by an H atom. This of course also applies for the preparation of labeled tyrosine (R¹(O) replaced by H) and labeled m-hydroxy-phenylalanine (R²(O) replaced by an H). A hydrolytic cleaving-off of the mentioned H atoms in step (5) is not necessary in these cases, i.e. for example for preparing labeled phenylalanine step (5) is completely not applicable. In this last case the following applies: R¹(O)═R²(O)═R⁵═R⁶═H.

Against this background the present invention is also realized by the use of an L-enantiomerically pure labeling precursor as provided in step (1) of the method according to the invention, for preparing [¹⁸F]fluorine labeled aromatic L-amino acids.

Therefore, the present invention is a renunciation of the current approach in the art. With the new method the time-consuming separation of the enantiomers is cancelled, which is inevitable in the art to obtain the labeled L-amino acid. That is why the method according to the invention provides a simplified synthesis, whereby a maximum of stereo-chemical purity of the [¹⁸F]fluorine labeled aromatic L-amino acid is ensured. This is especially crucial for an intended use of the [¹⁸F]fluorine labeled aromatic L-amino acid in a living organism, since only chirally pure L-enantiomeric amino acids can enter biological cells and, if applicable, are metabolized therein.

Furthermore, the method according to the invention is completely automatable and thereby ensuring a high product quality in a reproducible manner. In addition, the well-controllable radiation emission which is herewith connected means an increase of security for the laboratory staff.

By means of the method according to the invention several aromatic L-amino acids can be prepared in a reliable manner.

The method according to the invention is especially characterized by its short synthesis time period. The preparation of 6-[¹⁸F]fluoro-3,4-dihydroxy-L-phenylalanine ([¹⁸F]DOPA) can be managed within a time period of approximately 45 minutes, whereas the preparation of this amino acid according to the nucleophilic substitution via [¹⁸F]fluorine as described in the art takes approximately 110 minutes; cf. for this Lemaire C. et al. (1993), Appl. Radiat. Isot., Vol. 44, No. 4, 737-744.

The method according to the invention can be performed in any appropriate reaction medium that is described for fluoride labeling, such as acetonitrile, dimethyl sulfoxide, benzonitrile, dimethyl formamide.

The L-enantiomeric labeling precursor which is provided in step (1) can be prepared by means of generally known methods of the organic chemistry. Various organic-chemical protecting groups for hydroxy groups (R¹, R²), amino functions (R³) or carboxy functions (R⁴), respectively, can be used as the substituents R¹-R⁴. These protecting groups as well as methods for attaching or removing them are generally known in the art; cf. for this e.g. Theodora W. Green, Peter G. M. Wuts, “Protective Groups in Organic Synthesis”, 3^{rd} Edition (1999), John Wiley & Sons Inc., ISBN 0-471-160119-9.

Therefore, the labeling precursor which is crucial for the method according to the invention is also a subject-matter of the present invention, whereby the following applies: Z═CHO and Y═NO₂.

For the method according to the invention it is preferred if R¹ and R² are each selected from the group consisting of: CH₃, CH₂OCH₃, CH₂OCH₂(C₆H₅), CH₂SCH₃, CH₂SCH₂(C₆H₅), CH₂OCH₂Cl, CH₂OCH₂Br, CH₂COC₆H₃-3,4-Cl, CH₂COC₆H₃-2,6-Cl₂, CH₂═CH₂, CH(CH₃)₂, c-C₆H₁₁, C(CH₃)₃, CH₂C₆H₅, 2,6-(CH₃)₂C₆H₃CH₂, 4-CH₃OCH₄CH₂, o-NO₂-C₆H₄CH₂, (CH₃)₂NCOC₆H₄CH₂, COCH₃, COC₆H₅, CO₂CH₃, COOCH₂CCl₃, CONHPh (Ph=phenyl) and CONH-i-Bu (Bu=butyl).

The use of these protecting groups has the advantage that especially well-established and easily attachable and removable protecting groups are provided. As mentioned, in the case of preparing [¹⁸F]fluorine labeled tyrosine only the protecting group R¹ at position four of the benzene ring, and in case of [¹⁸F]fluorine labeled m-tyrosine only the protecting group R¹ at the position five of the benzene ring is required, and in case of [¹⁸F]fluorine labeled phenylalanine no protecting groups are required. For the preparation of these three before-mentioned [¹⁸F]fluorine labeled aromatic L-amino acids only R² (preparation of 2-[¹⁸F]fluoro-L-tyrosine), or R¹ (preparation of 2-[¹⁸F]fluoro-meta-L-tyrosine) is cleaved off hydrolytically or step (5) is cancelled completely (preparation of 2-[¹⁸F]fluoro-L-phenylalanine). For the preparation of [¹⁸F]DOPA both R¹ as well as R² have to be provided as protecting groups which are again cleaved-off in step (5) in accordance with the invention.

The variety of the preferred protecting groups also demonstrates the multi-purpose applicability of the method.

According to another preferred alternative R¹ and R² are a cyclic acetal, which is preferably selected from the group consisting of: methylene, dimethylmethylene, cyclohexylidene, diphenylmethylene, ethoxymethylene acetal and cyclic boric acid ester.

This measure has the advantage that well-established, easily attachable and removable protecting groups are provided which can especially be used for the preparation of FDOPA. In the DOPA molecule at the benzene ring thereof two OH groups are located in ortho position in relation to each other. The acetal protecting group in this connection forms a methylene bridge between the two OH groups. In the following, by the matter of example, a labeling precursor of step (1) is shown, whereby R¹ and R² are a methylene acetal:

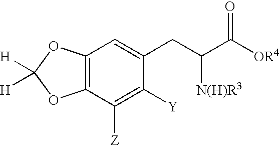

For the method according to the invention it is furthermore preferred, if Z is a substituent of second order, which is preferably selected from the group consisting of: CHO, NO₂, SO₂Me (Me=methyl), NR₃⁺ (R=alkyl group), CF₃, CN, COR (R=alkyl/aryl), COOH, Br, Cl and I.

According to this preferred embodiment for Z basically all substituents of “second order” are applicable. In this connection Z serves for the positivation of the carbon atom comprising the leaving group Y and placed in ortho position to the adjacent Z, by means of which the attachment of the negatively charged fluoride to this C atom is facilitated, and after the leaving of Y⁻ the corresponding fluorine compound is created. This preferred measure has therefore the advantage that a substituent comprising a sufficiently high electron-attracting potential is provided, which is, as a result, well-qualified as a so-called activating group. The mentioned preferred substituents can be rapidly and easily removed by means of methods which are known in the art.

In case the electron-attracting group Z is an aldehyde group (CHO) it is preferred, if step (3) is performed by the aid of a decarbonylization catalyser, preferably a Tris(triphenylphosphin)-rhodium(I)-chloride and/or concentrated sulphoric acid (H₂SO_{4conc.}) and/or palladium on activated carbon (Pd/C) and/or a Wilkinson catalyser (rhodium catalyser).

This measure has the advantage that on account of the decarbonylization catalyser a precise cleaving-off of the aldehyde group is enabled without e.g. a cleaving-off of the added [¹⁸F]fluoride ion.

Furthermore, it is preferred if Y is a substituent which is selected from the group consisting of: F, NO₂, OTs (=tosyl/toluene sulfone acid ester), Cl, Br, I, N₃ and NR₃⁺ (R=alkyl/aryl).

This has the advantage that also with this measure a leaving group for a nucleophilic substitution at the aromatic compound and a leaving group with sufficiently high cleaving-off tendency is provided, which is established in the art.

In a preferred further development of the method according to the invention R³ is/are one or several substituent(s) which is/are selected from the group consisting of: 9-fluorenylmethylcarbamate, CO₂CH₂CCl₃, CO₂CH₂CH₂Ph (Ph=phenyl), CO₂C(CH₃)CHBr₂, CO₂C(CH₃)₂CCl₃, CO₂C(CH₃)₃, N-hydroxypiperidinylcarbamate, CO₂CH₂Ph (Ph=phenyl), CO₂CH₂-p-CH₃OC₆H₄, CO₂CH₂-p-NO₂C₆H₄, CHO; COCH₃, COCH₂Cl, COCCl₃, COCF₃, COC₆H₅, phthalimide, dithiasuccinimide, N-5-dibenzosuberylamine, N-1,1-dimethylthiomethylene, N-benzylidene, N-1,3-dithiolan-2-ylidene, N-diphenylmethylene, ═CHN(CH₃)₂ and ═CN(CH₂C₆H₅)₂.

Furthermore, it is preferred if R⁴ is/are one or several substituent(s) which is/are selected from the group consisting of: CH₃, C₂H₅, CH₂OCH₃, CH₂SCH₃, CH₂OCH₂C₆H₅, CH₂CCl₃, C(CH₃)₃, CH₂C₆H₅, CH₂C₆H₂-2,4,6-(CH₃)₃.

The two afore-said measures have the advantage that protecting groups for the amino function or the carboxyl function, respectively, are used which are suitable and well-established in the art, and which are easily attachable and sufficiently easily cleavable.

According to a preferred embodiment of the method according to the invention all steps are performed in a single reaction vial.

This measure has the advantage that an especially simple handling of the method is reached, whereby a contamination of further reaction vials is avoided. This means an additional security for the laboratory staff. Furthermore, this measure enables an easy automatization of the method according to the invention.

According to a preferred embodiment of the method according to the invention all steps take place automized, preferably by the aid of a synthesis robot, further preferred by the aid of a compact synthesis apparatus.

This measure enables the realization of the method according to the invention in form of a high throughput method, so that the method can be used for routine production of [¹⁸F]fluorine labeled aromatic L-amino acids. Thereby also a high product quality and reproducibility is ensured. The use of a compact synthesis apparatus has the additional advantage that it takes up much smaller room in comparison to a conventional synthesis robot, resulting in a distinct reduction of the costs for e.g. a cell made of lead or a radiochemical laboratory, respectively.

According to a preferred embodiment of the method according to the invention, the following further step is performed:

(6) Preparation of a tracer by formulating the [¹⁸F]fluorine labeled aromatic L-amino acid with a pharmaceutically acceptable carrier and/or a solvent.

This embodiment has the advantage that the radioactive labeled amino acid is already provided in such a form that it can be directly applied into a living organism within the PET method mentioned at the outset. Pharmaceutically acceptable carriers or solvents, respectively, are generally known in the art; cf. Bauer, Frömming, Führer, Lehrbuch der pharmazeutischen Technologie [Textbook of the Pharmaceutical Technology], 6th edition, 1999, Wiss. Verl.-Ges., Stuttgart.

Against the background of the potential of the [¹⁸F]fluorine labeled aromatic L-amino acid produced according to the invention, and the advantages which emerge therefrom and as discussed here before and at the outset, subject-matter of the present invention also is a method for preparing a diagnostic agent comprising the following steps:

(1) providing a [¹⁸F]fluorine labeled aromatic L-amino acid, and

(2) formulating the amino acid of step (1) with a pharmaceutically acceptable carrier and/or a solvent and, if applicable, further pharmaceutical excipients,

whereby step (1) is performed by means of the method according to the invention as described before. It is herewith preferred if the diagnostic agent is designated for the use within the positron emission tomography (PET).

A further subject-matter of the present invention is a method for visualizing metabolic processes comprising the following steps:

(1) providing a [¹⁸F]fluorine labeled aromatic L-amino acid;

(2) introducing the amino acid of step (1) into a living organism, and

(3) detection of the introduced amino acid in the living organism,

whereby step (1) is performed by means of the method according to the invention as described before. It is herewith preferred if step (3) takes place by means of a radiation detector, preferably a positron emission tomography scanner and/or by the aid of autoradiography.

It goes without saying that the features named above and those still to be explained below can be used not only in the respectively specified combination, but also in other combinations or on their own without departing from the scope of the present invention.

The present invention is now illustrated by means of embodiments which are of pure exemplary character and do not narrow the invention.

### DETAILED DESCRIPTION OF THE PREFERRED EMBODIMENT

### EXAMPLE 1

### Preparation of the L-Enantiomeric Labeling Precursor

The preparation of the labeling precursors is outlined in the following by means of a flow-chart. As substituents the following was chosen: Z═CHO and Y═NO₂.

A) [¹⁸F]-Phenylalanine Precursor

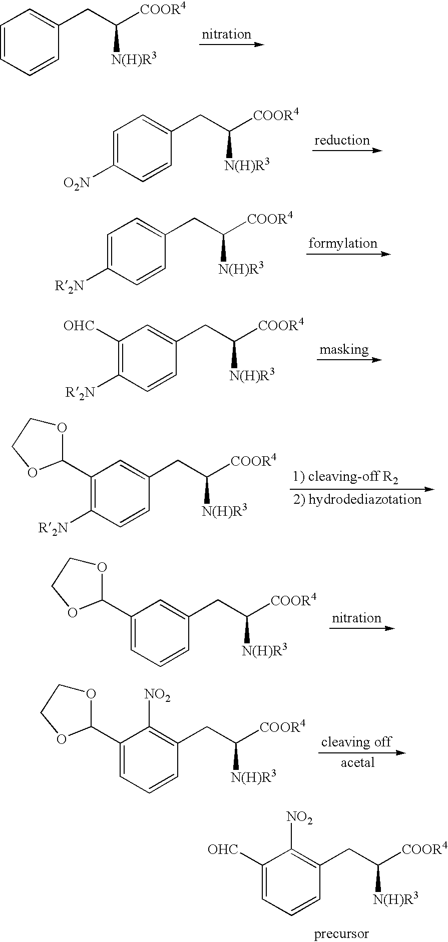

B) [¹⁸F]-Tyrosine Precursor

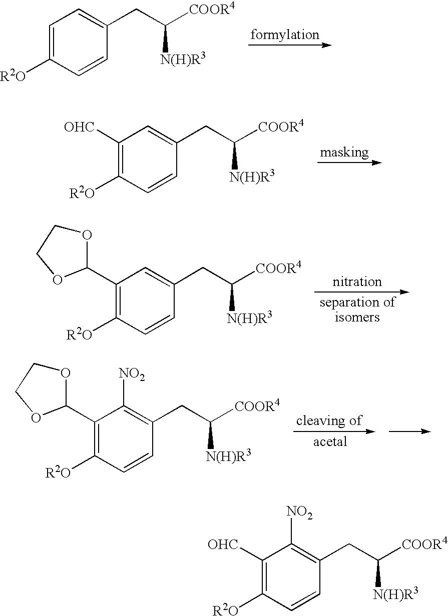

C) [¹⁸F]-5-Hydroxy-Phenylalanine Precursor

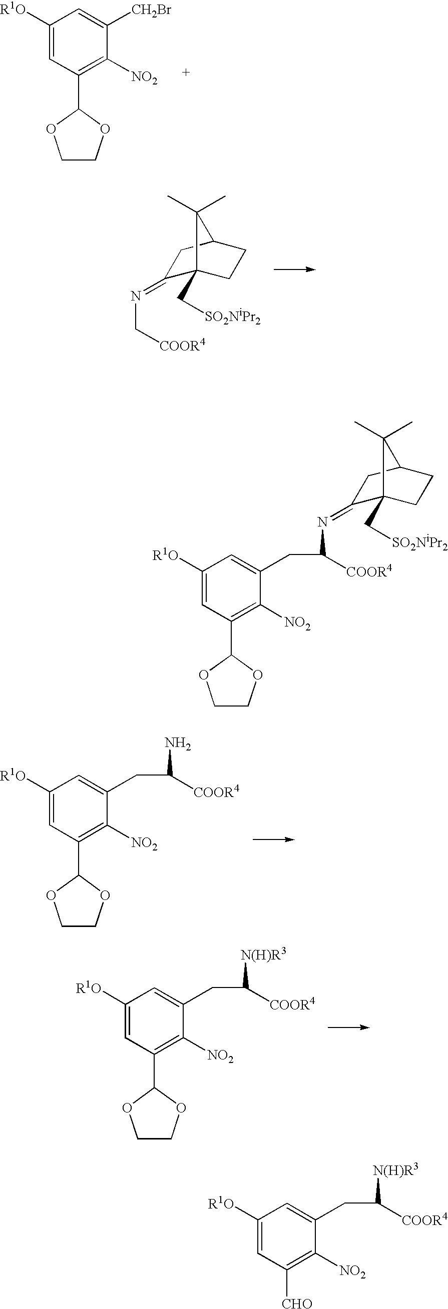

D) [¹⁸F]-DOPA Precursor

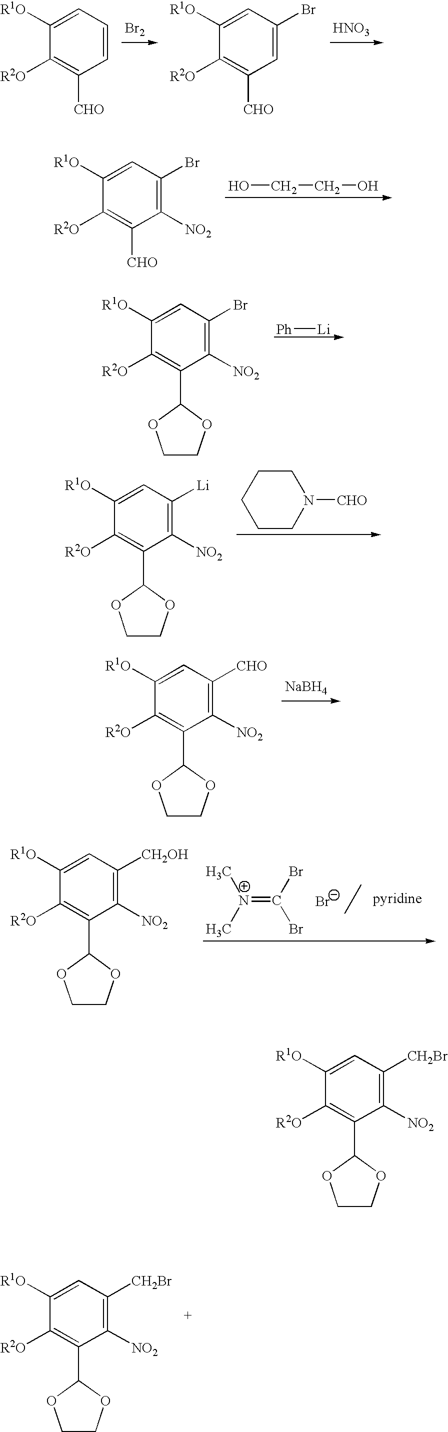

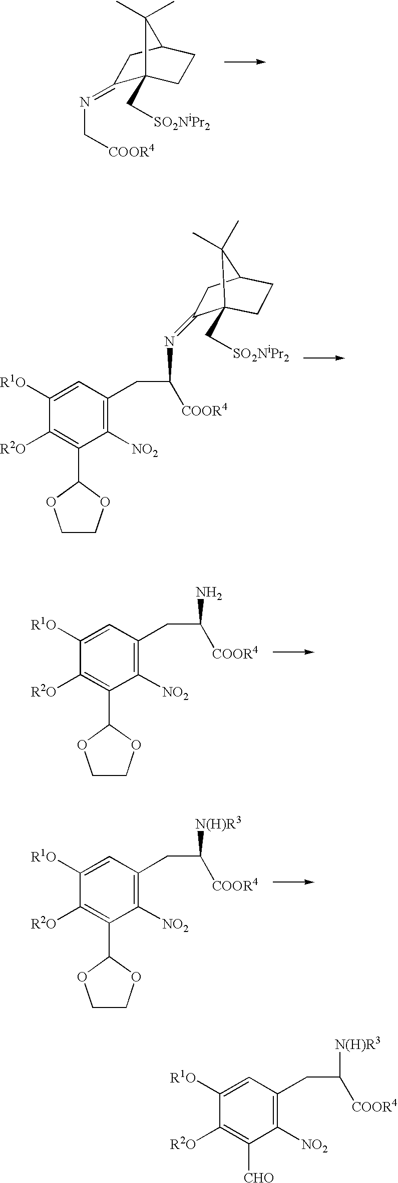

### EXAMPLE 2

### Substitution of the Leaving Group Y for the [¹⁸F]Fluoride Ion

In the first step of the actual synthesis reaction the substituent Y, i.e. the leaving group, is substituted for an [¹⁸F]fluoride ion according to methods which are generally known in the art:

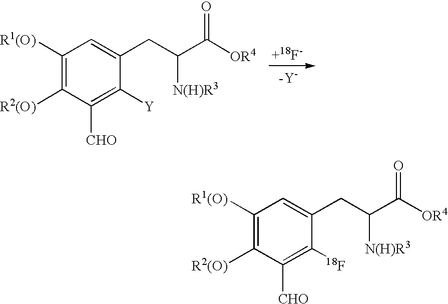

Due to the high electron density in the benzene ring, which is additionally increased by the preparation of labeled L-tyrosine (R¹(O)═H, R²(O)═OH) and L-DOPA (R¹(O)═R²(O)═OH) on account of the hydroxyl groups, the highly electron attracting group CHO (activating group) in direct vicinity to the leaving group Y facilitates this substitution.

### EXAMPLE 3

### Cleaving-Off of the Activating Group CHO

In the following step the cleaving-off of the activating aldehyde group is taken place. This can be precisely and easily realized by means of a decarbonylization catalyser (Tris(triphenylphosphin)-rhodium(I)-chloride and/or a Wilkinson catalyser):

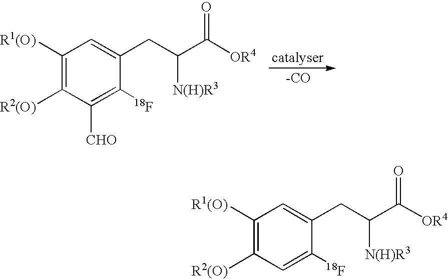

### EXAMPLE 4

### Hydrolytic Cleaving-Off of the Protecting Groups R³, R⁴

In the next step the cleaving-off of the protecting groups R³ and R⁴ by means of hydrolysis occurs according to methods which are generally known in the art:

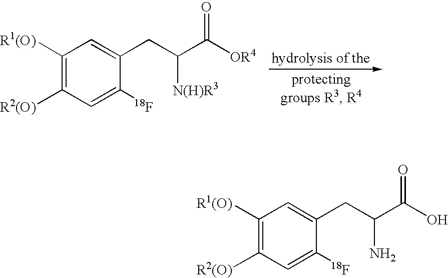

### EXAMPLE 5

### Hydrolytic Cleaving-Off of R¹ and R²

For the preparation of 2-[¹⁸F]fluoro-L-phenylalanine this step is not required since the following applies: R¹(O)═R²(O)═H. In case R¹(O) and R²(O) do not correspond to a hydrogen atom in this last synthesis step which is simultaneously performed with the preceding cleaving-off of R³ and R⁴, the cleaving-off of these protecting groups R¹(O) and R²(O) together with a transfer into OH groups, i.e. for preparing 2-[¹⁸F]fluoro-L-tyrosine R²(O) is transferred into R⁶═OH, whereby the following applies: R¹(O)═R⁵═H. In the case of the preparation of 2-[¹⁸F]fluoro-5-hydroxy-L-phenylalanine the following applies: R²(O)═R⁶═H and R¹ is transferred into R⁵═OH. For the preparation of 6-[¹⁸F]fluoro-3,4-dihydroxy-L-phenylalanine (FDOPA) R¹(O) is transferred in R⁵═OH and R²(O) is transferred in R⁶═OH:

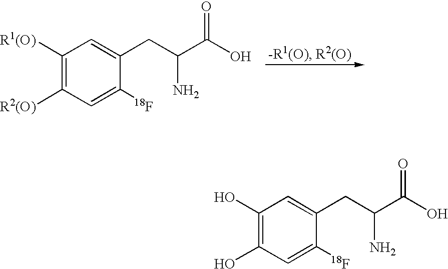

In case of the other compounds to be prepared, the individual reactions are generally known in the art.

What is claimed is:

1. A method for preparing [¹⁸F]fluorine labeled aromatic L-amino acids comprising the following steps:
(1) providing the following L-enantiomeric compound in an appropriate reaction medium: 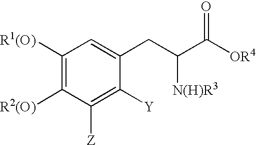
wherein
R¹ and R² are appropriate protecting groups for OH groups, provided a hydroxy amino acid is to be prepared;
Z is an electron-attracting group;
Y is a leaving group for a nucleophilic substitution; w
R³ is one or several appropriate protecting groups for an amino function; and
R⁴ is an appropriate protecting group for a carboxyl group;
(2) performing nucleophilic substitution of Y for a negatively charged [¹⁸F]fluorine ion to prepare the following compound: 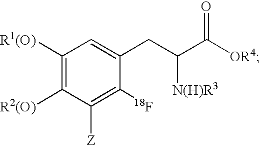
(3) cleaving-off of Z to prepare the following compound: 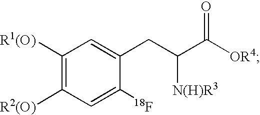
(4) hydrolytic cleaving-off of R³ and R⁴ to prepare the following compound: 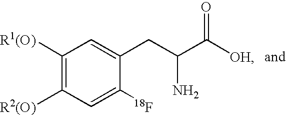
(5) hydrolytic cleaving-off of R¹ and R² to prepare the following [¹⁸F]fluorine labeled aromatic L-amino acid: 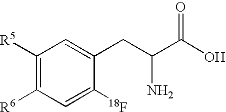
wherein R⁵ and R⁶ substituents are in each case an H atom or an OH group.

2. The method according to claim 1, wherein R¹ and R² are selected from the group consisting of: CH₃, CH₂OCH₃, CH₂OCH₂(C₆H₅), CH₂SCH₃, CH₂SCH₂(C₆H₅), CH₂OCH₂Cl, CH₂OCH₂Br, CH₂COC₆H₃-3,4-Cl, CH₂COC₆H₃-2,6-Cl₂, CH₂═CH₂, CH(CH₃)₂, c-C₆H₁₁, C(CH₃)₃, CH₂C₆H₅, 2,6-(CH₃)₂C₆H₃CH₂, 4-CH₃OCH₄CH₂, o-NO₂-C₆H₄CH₂, (CH₃)₂NCOC₆H₄CH₂, COCH₃, COC₆H₅, CO₂CH₃, COOCH₂CCl₃, CONHPh (Ph=phenyl), and CONH-i-Bu (Bu=butyl).

3. The method according to claim 1, wherein R¹ and R² are a cyclic acetal.

4. The method according to claim 3, wherein the cyclic acetal is selected from the group consisting of: methylene, dimethylmethylene, cyclohexylidene, diphenylmethylene, ethoxymethylenacetal and cyclic boric acid ester.

5. The method according to claim 1, wherein Z is a substituent of second order.

6. The method according to claim 5, wherein the substituent of second order is selected from the group consisting of: CHO, NO₂, SO₂Me (Me=methyl), NR₃⁺ (R=alkyl group), CF₃, CN, COR (R=alkyl/aryl), COOH, Br, Cl and I.

7. The method according to claim 1, wherein Y is a substituent which is selected from the group consisting of: F, NO₂, OTs, Cl, Br, I, N₃ and NR₃⁺ (R=alkyl/aryl).

8. The method according to claim 1, wherein R³ is one or several substituents which are selected from the group consisting of: 9-fluorenylmethylcarbamate, CO₂CH₂CCl₃, CO₂CH₂CH₂Ph (Ph=phenyl), CO₂C(CH₃)CHBr₂, CO₂C(CH₃)₂CCl₃, CO₂C(CH₃)₃, N-hydroxypiperidinylcarbamate, CO₂CH₂Ph (Ph=phenyl), CO₂CH₂-p-CH₃OC₆H₄, CO₂CH₂-p-NO₂C₆H₄, CHO; COCH₃, COCH₂Cl, COCCl₃, COCF₃, COC₆H₅, phthalimide, dithiasuccinimide, N-5-dibenzosuberylamine, N-1,1-dimethylthiomethylene, N-benzylidene, N-1,3-dithiolan-2-ylidene, N-diphenylmethylene, ═CHN(CH₃)₂ and ═CN(CH₂C₆H₅)₂.

9. The method according to claim 1, wherein R⁴ is one or several substituents which are selected from the group consisting of: CH₃, C₂H₅, CH₂OCH₃, CH₂SCH₃, CH₂OCH₂C₆H₅, CH₂CCl₃, C(CH₃)₃, CH₂C₆H₅, CH₂C₆H₂-2,4,6-(CH₃)₃.

10. The method according to claim 1, wherein step (3) is performed by the aid of a decarbonylization catalyser.

11. The method according to claim 10, wherein the decarbonylization catalyser is selected from the group consisting of: Tris(triphenylphosphine)-rhodium(I)-chloride, concentrated sulphoric acid (H₂SO_{4conc.}), and palladium on activated carbon (Pd/C), and a Wilkinson catalyser (rhodium catalyser).

12. The method according to claim 1, wherein all steps are performed in a single reaction vial.

13. The method according to claim 1, wherein all steps are performed automatized.

14. The method according to claim 13, wherein all steps are performed by the aid of a compact synthesis apparatus.

15. The method according to claim 1, further comprising:
(6) preparing a tracer by formulating the [¹⁸F]fluorine labeled aromatic L-amino acid with a pharmaceutically acceptable carrier and/or a solvent.

16. A method for preparing a diagnostic agent comprising the following steps:
(1) providing a [¹⁸F]fluorine labeled aromatic L-amino acid, and
(2) formulating the amino acid of step (1) with a pharmaceutically acceptable carrier and/or a solvent and, if applicable, further pharmaceutical excipients,
wherein step (1) is performed by means of the method according to claim 1.

17. The method according to claim 16, wherein the diagnostic agent is designated for a use within the positron emission tomography (PET).

18. A method for visualizing metabolic processes comprising the following steps:
(1) providing of a [¹⁸F]fluorine labeled aromatic L-amino acid;
(2) introducing of the amino acid of step (1) into a living organism, and
(3) detecting the introduced amino acid in the living organism,
wherein step (1) is performed by the method according to claim 1.

19. The method according to claim 18, wherein step (3) is performed by means of a radiation detector.

20. L-enantiomeric compound, comprising the following chemical structure: 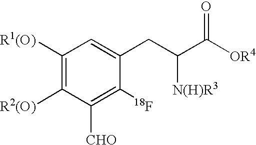
wherein
R¹ and R² are appropriate protecting groups for OH groups;
R³ is an appropriate protecting group for an amino function; and
R⁴ is an appropriate protecting group for a carboxyl function.

21. The L-enantiomeric compound according to claim 20, wherein R¹ and R² are each selected from the group consisting of: H, CH₃, CH₂OCH₃, CH₂OCH₂(C₆H₅), CH₂SCH₃, CH₂SCH₂(C₆H₅), CH₂OCH₂Cl, CH₂OCH₂Br, CH₂COC₆H₃-3,4-Cl, CH₂COC₆H₃-2,6-Cl₂, CH₂═CH₂, CH(CH₃)₂, c-C₆H₁₁, C(CH₃)₃, CH₂C₆H₅, 2,6-(CH₃)₂C₆H₃CH₂, 4-CH₃OCH₄CH₂, o-NO₂-C₆H₄CH₂, (CH₃)₂NCOC₆H₄CH₂, COCH₃, COC₆H₅, CO₂CH₃, COOCH₂CCl₃, CONHPh (Ph=phenyl) and CONH-i-Bu (Bu=butyl).

22. The L-enantiomeric compound according to claim 20, wherein R¹ and R² correspond to a cyclic acetal.

23. The L-enantiomeric compound according to claim 22, wherein the cyclic acetal is selected from the group consisting of: methylene, dimethylmethylene, cyclohexylidene, diphenylmethylene, ethoxymethylenacetal and cyclic boric acid ester.

24. The L-enantiomeric compound according to claim 20, wherein R³ is one or several substituents which are selected from the group consisting of: 9-fluorenylmethylcarbamate, CO₂CH₂CCl₃, CO₂CH₂CH₂Ph (Ph=phenyl), CO₂C(CH₃)CHBr₂, CO₂C(CH₃)₂CCl₃, CO₂C(CH₃)₃, N-hydroxypiperidinylcarbamate, CO₂CH₂Ph (Ph=phenyl), CO₂CH₂-p-CH₃OC₆H₄, CO₂CH₂-p-NO₂C₆H₄, CHO; COCH₃, COCH₂Cl, COCCl₃, COCF₃, COC₆H₅, phthalimide, dithiasuccinimide, N-5-dibenzosuberylamine, N-1,1-dimethylthiomethylene, N-benzylidene, N-1,3-dithiolan-2-ylidene, N-diphenylmethylene, ═CHN(CH₃)₂ and ═CN(CH₂C₆H₅)₂.

25. The L-enantiomeric compound according to claim 20, wherein R⁴ corresponds to one or several substituents which are selected from the group consisting of: CH₃, C₂H₅, CH₂OCH₃, CH₂SCH₃, CH₂OCH₂C₆H₅, CH₂CCl₃, C(CH₃)₃, CH₂C₆H₅, CH₂C₆H₂-2,4,6-(CH₃)₃.
